(19) United States
(12) Patent Application Publication
(10) Pub. No.: US 2025/0327739 A1
(43) Pub. Date: Oct. 23, 2025

(54) WATER QUALITY MEASUREMENT DEVICE

(71) Applicant: MIMBLY AB, Göteborg (SE)

(72) Inventors: Nicolas MAXANT, Göteborg (SE); Aron HAKONEN, Hisings Baka (SE); Sara EL HARKAOUI, Göteborg (SE)

(73) Assignee: MIMBLY AB, Göteborg (SE)

(21) Appl. No.: 18/864,772

(22) PCT Filed: May 12, 2023
(86) PCT No.: PCT/SE2023/050467

§ 371 Date: Nov. 11, 2024

(30) Foreign Application Priority Data
May 13, 2022 (SE) 2230143-6
Oct. 6, 2022 (SE) 2251155-4

(51) Int. Cl. G01N 21/25; G01N 21/59; G01N 33/18

(52) U.S. Cl. CPC G01N 21/256; G01N 21/59; G01N 33/1893

(57) ABSTRACT

A water quality measurement device for measuring water quality, the water quality measuring device comprising: a white light emitting element configured to emit substantially white light, an infra-red light emitting element configured to emit substantially infra-red light, and a light receiving element configured to receive and detect light from the white light emitting element and the infra-red light emitting element; wherein, the white light emitting element is arranged co-axial and opposite the light receiving element, and wherein the emission axis of the infra-red emitting element is arranged substantially orthogonal the incident axis of the light receiving element.

### FIELD OF THE INVENTION

The present disclosure relates to a water quality measurement device. In particular in relates to a water quality measurement device comprising a white light emitting element, an infra-red light emitting element, and a light receiving element.

### BACKGROUND OF THE INVENTION

Optoelectrical devices for measuring the quality of water are known in the field of measuring water turbidity in household appliances such as washing machines, dishwashers etc.

U.S. Pat. No. 8,531,670 B2 (EMZ Hanauer) describes an optical turbidity sensor for installation in a household washing machine or dishwasher having separate subspaces for the provision of water to be measured and the provision of the light-emitting and light-receiving elements.

It would be ideal if devices did not require installation into the water comprising compartment of washing devices as such installations lead to a point of failure where water may leak from the water comprising compartment.

Improved devices may furthermore be capable of detecting additional water quality parameters.

### SUMMARY OF THE INVENTION

Accordingly, the present invention preferably seeks to mitigate, alleviate or eliminate one or more of the above-identified deficiencies in the art and disadvantages singly or in any combination and solves at least the above mentioned problems by providing a water quality measurement device for measuring water quality, the water quality measuring device comprising: a white light emitting element configured to emit substantially white light, an infra-red light emitting element configured to emit substantially infra-red light, and a light receiving element configured to receive and detect light from the white light emitting element and the infra-red light emitting element; wherein, the white light emitting element is arranged co-axial and opposite the light receiving element, and wherein the emission axis of the infra-red emitting element is arranged substantially orthogonal the incident axis of the light receiving element.

A process for measuring water quality is also provided.

A process for calibrating the water quality measurement device is provided.

Further advantageous embodiments are disclosed in the appended and dependent patent claims.

### BRIEF DESCRIPTION OF THE DRAWINGS

These and other aspects, features and advantages of which the invention is capable will be apparent and elucidated from the following description of embodiments of the present invention, reference being made to the accompanying drawings, in which

FIG. 1 is a schematic top view of a water quality measurement device according to an aspect.

FIG. 2 is a schematic side view of a water quality measurement device according to an aspect.

FIG. 3 is a schematic side view of a water quality measurement device according to an aspect.

FIG. 4 is a schematic top view of a water quality measurement device according to an aspect. Water to be measured is shown as the dotted region.

FIG. 5 is a schematic top view of a water quality measurement device showing the intensities α_{A}, α_{B}, sensor sensitivities β_{A}, β_{B}, interface loss terms γ_{LA}, γ_{LB} water transmissivity λ_{T}, and water diffractivity λ_{D}.

### DETAILED DESCRIPTION

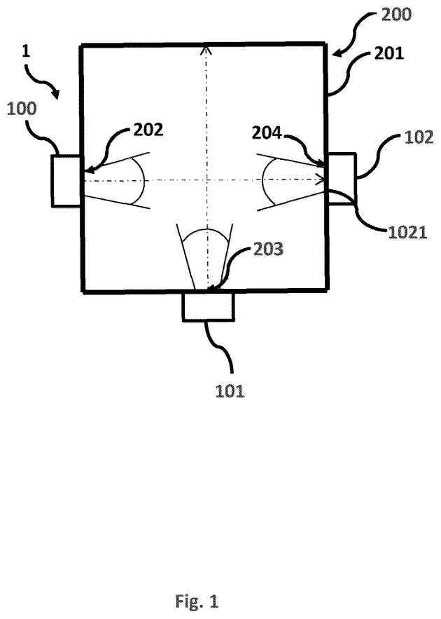

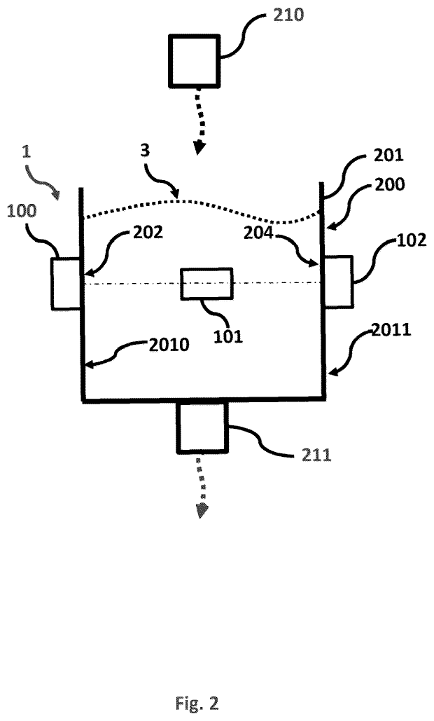

FIG. 1 shows a water quality measurement device 1 comprising a white light emitting element 100, an infra-red light emitting element 101, and a light receiving element 102. The light receiving element 102 is arranged to receive light from the white light emitting element 100, and the infra-red light emitting element 101.

The white light emitting element 100 is arranged co-axial and opposite the light receiving element 102. The white light emitting element 100 emits light toward the light receiving element 102. The light emitted from the white light emitting element 100 travels in a substantially straight path to the light receiving element 102. No reflectors or mirrors are provided to the device 1. The emission axis of the infra-red light emitting element 101 is substantially orthogonal the incident axis of the light receiving element 102. The path of undisturbed or un-scattered light emitted from the white light emitting element 100 and the infra-red light emitting element 101 are shown in FIG. 1 by the dashed lines with arrows indicating the direction of light travel.

The emission axis of the infra-red light emitting element 101 is arranged substantially orthogonal the incident axis of the light receiving element 102. The infra-red light emitting element 101 is not co-axial to the light receiving element 102. The term substantially orthogonal as used herein means that the angle between the optical incident axis of the light receiving element 102 and the optical emission axis of the infra-red light emitting element 101 is from about 85° to about 95°, such as about 90°. The infra-red light emitting element 101 emits infra-red light in a direction substantially orthogonal to the axis formed between the white light emitting element 100 and the light receiving element 102. In such an arrangement only infra-red light scattered, absorbed or otherwise disturbed after leaving the infra-red light emitting element 101 is received at the light receiving element 102. If there is no disturbance, absorbance or scattering of the light emitted from the infra-red light emitting element 101 no, or very little infra-red light is received at the light receiving element 102.

As shown in FIG. 1, and is known within the field, each of the white light emitting element 100 and infra-red light emitting element 101 emit a cone of light, and therefore the optical emission axis is the centre axis of the emitted cone of light. Similarly, the light receiving element 102 has a viewing angle and receives incident light at an angle greater than 0° with respect to the central incident axis. The term light incident axis as used herein refers to the central incident axis.

The white light emitting element 100 ideally emits light across the wavelength spectrum from about 400 nm to about 750 nm. The infra-red light emitting element 101 may emit infra-red light in the infra-red spectrum from about 700 nm to about 1 mm, such as about 750 nm to about 900 nm. The white light emitting element 100 ideally is a single LED emitting light across the spectrum from about 400 nm to about 750 nm. The broad spectrum emitted from the white light emitting element 100 enables improved colour and turbidity measurement by a single light source. By emitting a broad spectrum with a single light source, a range of colours of the water can be measured, furthermore fine turbidity measurement can be performed without the need for additional components, and therefore a smaller total device 1 size.

The light receiving element 102 is ideally a light sensor configured to receive and detect light across a broad spectrum. The light receiving element 102 generally receives and detects light separately in the infra-red, red, green and blue spectrums. That is, the intensity of received light for each spectrum is measurable and can be read from the element 102 separately. The light receiving element 102 may also receive, detect and output a measured value for received light across the full receivable spectrum i.e., infra-red and white, in an additional channel. The separate detection spectrums enable improved colour and turbidity measurement. The light receiving element 102 may receive and detect light in the wavelength range from about 400 nm to about 1000 nm, such as from about 400 nm to about 900 nm. The light receiving element 102 may be an ambient light sensor configured to receive and detect infra-red, red, green and blue light. Ideally, the ambient light sensor is configured to detect red, green, blue and white light, where white light corresponds to broad spectrum detection over at least infra-red, red, green and blue wavelength spectrums. The light receiving element 102 may comprise an electrically implemented filter for determining the intensity of received light in a respective colour. Both the white light emitting element 100 and the infra-red light emitting element 101 may emit light into a volume of water, such as processed wash water in order to detect the quality of the water.

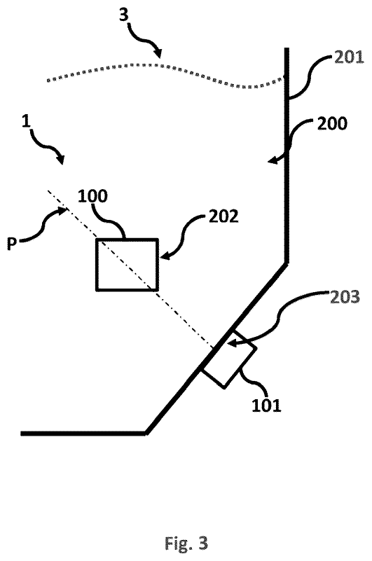

The white light emitting element 100, infra-red light emitting element 101 and the light receiving element 102 may be in some instances arranged in the same plane. In FIG. 1 the plane is a horizontal plane, however, the plane need not be horizontal. The plane referred to is the plane formed by the optical axes of the white light emitting element 100, infra-red light emitting element 101, and the theoretical incident axis of the light receiving element 102. In FIG. 3 the plane is marked as P and is formed along the dashed line and extends into the page. In FIG. 3 the white light emitting element 100 and the infra-red light emitting element 101 are shown, the light receiving element 102 is not shown as it is behind and co-axial the white light emitting element 100. The optical axis of the white light emitting element 100 is into the page in FIG. 3.

The white light emitted from the white light emitting element 100 traverses the volume of water and is received at the light receiving element 102. The white light emitted from the white light emitting element 100 has a substantially known wavelength spectrum and known intensity over the known spectrum. As the white light emitted from the white light emitting element 100 traverses the water volume the light is substantially filtered and the spectrum and intensity over the spectrum of the emitted white light is altered such that the received light at the light receiving element 102 has different intensities at different wavelengths compared to the white light emitted from the white light emitting element 100. The altered spectrum of white light received at the light receiving element 102 may be compared to the known emitted spectrum to determine water quality. For example, the colour of the water volume may be determined by comparing the wavelength spectrum, and intensity at various wavelengths of the received light at the light receiving element 102 to the emitted white light from the white light emitting element 100. If the volume of water has a certain colour this can be determined by comparing the relative intensities of the colours received at the light receiving element 102. For example, if the water is coloured red, due to for example a dye in the water, the red-coloured water will absorb light in the blue and/or green spectrum, leading to relatively lower intensities of blue and/or green light received at the light receiving element 102. Similarly, green coloured water results in a reduction of the blue spectrum received at the light receiving element 102.

The infra-red light emitting element 101 emits infra-red light into the volume of water to determine water quality. Infra-red light emitted from the infra-red light emitting element 101 traverses the volume of water and is at least partially scattered by the water, orthogonal to the light receiving element 102. A portion of the scattered light is received at the light receiving element 102. The portion of light received at the light receiving element 102 is correlated to the amount of scattering due to the water. Water having a higher turbidity scatters more light, and therefore the portion of light received at the light receiving element 102 corresponds to the level of turbidity of the water. That is, the more light received at the light receiving element 102 from the infra-red light emitting element 101, the greater the turbidity of the water.

The water quality measurement device 1 may comprise a receptacle 200 for receiving a volume of fluid 3, such as wash water. The receptacle 200 receives a volume of water therein for a duration. The receptacle may be defined by at least one wall 201. The white light emitting element 100, infra-red light emitting element 101 and the light receiving element 102 are directed into the receptacle 200. The light emitted from the white light emitting element 100 is directed into the receptacle 200. The light emitted from the infra-red light emitting element 101 is directed into the receptacle 200. The light receiving element 102 has a sensor region 1021 which is directed into the receptacle. The light receiving element 102 receives light which has been emitted into the receptacle. When fluid 3 is provided to the receptacle 200, the light from the white light emitting element 100 and/or the infra-red light emitting element 101 is emitted into the volume of fluid 3. When fluid 3 is provided to the receptacle 200, the light receiving element 102 receives light which has passed through the fluid.

As stated above the path white light travels from the white light emitting element 100 to the light receiving element 102 is substantially straight and free from mirrors or reflecting elements. As would be understood by the skilled person, some minor refraction to the light may occur due to the volume of water, and the wall 201 of the receptacle 200.

The white light emitting element 100, infra-red light emitting element 101 and the light receiving element 102 are provided external to the receptacle 200. That is, they are not provided within the receptacle and do not mechanically disturb the fluid within the receptacle 200. The white light emitting element 100, infra-red light emitting element 101 and light receiving element 102 may be separated from the volume of fluid within the receptacle 200 by the at least one wall 201. By providing the light emitting elements 100, 101 and the light receiving element 102 separate and external to the volume of fluid the white light emitting element 100, the infra-red light emitting element 101 and the light receiving element 102 are not subject to fouling, wear or other similar disturbances which may reduce the performance of the water quality measurement device 1. Additionally, by providing the white light emitting element 100, infra-red light emitting element 101 and the light receiving element 102 external to the water receptacle 200, the white light emitting element 100, infra-red light emitting element 101 and the light receiving element 102 are protected from damage due to contact with high temperature water.

The receptacle 200 has an inlet 210 at which water enters the receptacle 200. The receptacle 200 has an outlet 211 at which water exits the receptacle 200. The outlet 211 is separate to the inlet 210, that is, water is displaced through the receptacle 200. The receptacle is a flow-through receptacle 200, where the inlet 210 is separate to the outlet 211.

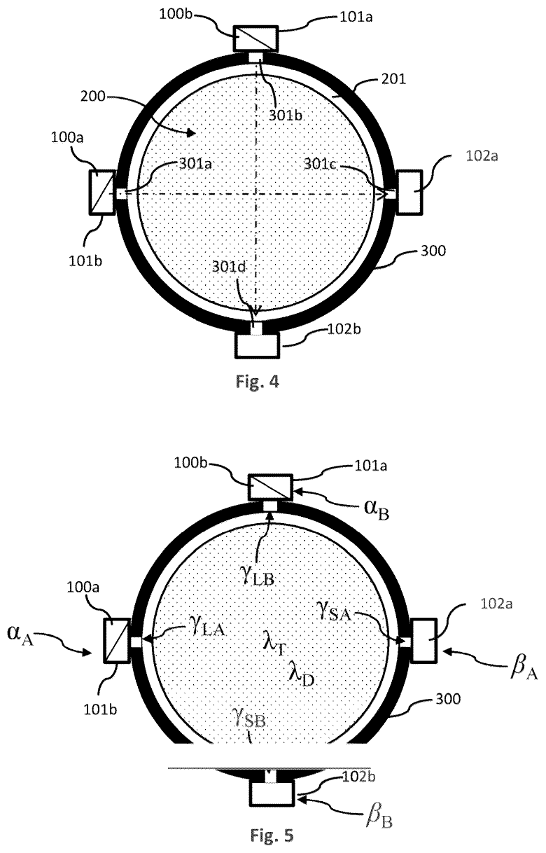

As shown in FIG. 4, the device 1 may comprise at least two white light emitting elements 100a, 100b. The device 1 may comprise at least two infra-red light emitting elements 101a, 101b. The device 1 may comprise at least two light receiving elements 102a, 102b. Each of the white light emitting elements 100a, 100b are substantially co-axial and opposite a respective light receiving element 102a, 102b. Each of the infra-red light emitting elements 101a, 101b are arranged such that their respective light emission axes are orthogonal the incident axes of a respective light receiving element 102a, 102b. The advantages of having such a plurality of white and infra-red light emission and light receiving will be detailed below.

In the device 1 with at least two white 100a, 100b, at least two infra-red 101a, 101b, light emitting elements the first white light emitting element 100a may be substantially co-located with the second infra-red light emitting element 101b. The second white light emitting element 100b may be substantially co-located with the first infra-red light emitting element 101a. Such an arrangement enables fewer light paths through the receptacle 200 compared to separate locations for each element, and as the elements 100a, 100b, 101a, 101b may be surface mounted LEDs, reduces manufacturing costs and space required for the elements.

The light receiving elements 102a, 102b may be arranged such that their respective light incident aces are substantially orthogonal. As described above, the elements 100a, 100b, 101a, 101b, 102a, 102b are typically arranged in a single plane. The device 1 does not comprises reflectors or mirrors. The transmission axes of the light emitting elements 100a, 100b, 101a, 101b are directed towards the receptacle.

The receptacle 200 may be a tube. The at least one wall 201 may therefore be a tubular wall 201 forming a tube. The at least one white light emitting element 100a, 100b, the at least one infra-red light emitting element 101a, 101b, and the at least one light receiving element 102a, 102b are provided around the circumference of the tubular wall.

As described above, the device 1 may comprise a plurality of light emitting and receiving elements 100a, 100b, 101a, 101b, 102a, 102b. By using a plurality, such as two: white light emitting elements 100a, 100b, two infra-red light emitting elements 101a, 101b, and two light receiving elements 102a, 102b, the ability to cancel out nonidealities at a relatively low cost is achieved. The plurality allows for self-detecting and compensating for absorbed light along the receptacle 200/water interface.

An illustration of the modelled unknown parameters, including error term F is shown in FIG. 5. Assuming a first light emitting element 100a, a first infra-red light emitting element 101a, and a first light receiving element 102a this gives:

$${β}_{A} = {α}_{A} {γ}_{LA} {λ}_{T} {γ}_{SA}$$

$${β}_{B} = {α}_{A} {γ}_{LA} {λ}_{D} {γ}_{SB}$$

And with two light emitting and receiving elements:

$${β}_{A}^{′} = {α}_{B} {γ}_{LB} {λ}_{D} {γ}_{SA}$$

$${β}_{B}^{′} = {α}_{B} {γ}_{LB} {λ}_{T} {γ}_{SB} .$$

Here β_{A} and β_{B} are the measured light intensities as reported by the first light receiving element 102a, and second light receiving element 102b respectively, α_{A} and α_{B} the emission intensities for the first white light emitting element 100a and the co-located second infra-red emitting element 101a, and the second white light emitting element 100b and the co-located first infra-red emitting element 101b respectively. γ_{LA} and γ_{LB} are the lumped terms corresponding to the light emitting elements' 100a, 100b, 101a, 101b, efficiency and emitting element-side pipe fouling (i.e., ≥0), γ_{SA} and γ_{SB} are lumped terms corresponding to receiving element-side pipe fouling and receiving element 102a, 102b sensitivities (i.e., ≥0), and λ_{T} and λ_{D} are the transmissivity and diffractivity of the water sample respectively. In the above α is controlled, β is measured, and γ_{LA}, γ_{LB}, λ_{T} and λ_{D} are unknowns. For the purpose of measuring the turbidity, the goal is to measure λ_{D}/λ_{T}, i.e., a relative measure of diffractivity, despite the presence of the unknowns γ_{LA}, γ_{LB}. Importantly, the addition of the second white light emitting element 100b, second infra-red light emitting element 101b, and second light receiving element 102b allows for the cancelling out the influence of the unknown γ_{LA}, γ_{LB} terms. That is, cancelling out the potential fouling on the pipe walls.

The turbidity measurement with at least two white light emitting elements 100a, 100b, at least two infra-red light emitting elements 101a, 101b is given by

$$n = \sqrt{\frac{{β}_{B}}{{β}_{A}} \cdot \frac{{β}_{A}^{′}}{{β}_{B}^{′}}}$$

Where BB/BA is the ratio of diffractive to transmissive measurements with both the first light emitting and receiving element 100a, 101a, 102a active and β_{B}/β_{A} is the ratio of diffractive to transmissive measurements with the second light emitting and receiving elements 100b, 101b, 102b active. It can be derived that the expression for the ratiometric turbidity measure is independent of the fouling terms is the geometric mean of the two ratiometric measurements. Fully cancelling out the fouling terms is made possible by having both first and second light emitting and receiving elements 100a, 100b, 101a, 101b, 102a, 102b.

The device 1 may be provided with a sleeve 300. The sleeve 300 is manufactured from an optically opaque material. That is, a material which is substantially non-transmissive to both white and infra-red light. The sleeve 300 surrounds the wall 201. Generally, the sleeve 300 substantially abuts the at least one wall 201 to reduce potential light leakage. If the receptacle 200 is a tube, then the sleeve 300 is also tubular. The sleeve 300 is provided with a plurality of slots 301a, 301b, 301c, 301d. The slots correspond to the emission and incidence axes for the light emitting and receiving elements 100a, 100b, 101a, 101b, 102a, 102b. The slots 301a, 301b, 301c, 301d are cavities within the sleeve 300 which enable light to pass unobstructed through the sleeve 300. If each white light emitting element 100a, 100b is co-located with a respective infra-red light emitting element 101b, 101a, then the number of slots 301a, 301b, 301c, 301d is less than the total number of light emitting and receiving elements. As shown in FIG. 4, the sleeve 300 may comprise four slots. Each slot 301a, 301b, 301c, 301d is aligned and opposite a corresponding slot 301c, 301d, 301a, 301b, forming a pair.

Water, such as wash water, flows into the device 1, specifically, water flows into the receptacle 200 at a flow rate. Water may be received within the device 1, specifically the receptacle 200, and partially, or fully fill the receptacle 200. The flow velocity of water within the receptacle 200 may be lower than the flow velocity of water at the inlet 210 to the receptacle 200. That is, the velocity of water may be reduced on entering the receptacle 200. This enables improved measuring performance.

Water flowing into the device 1 may flow at a flow rate of less than about 20 L/min, such as less than about 10 L/min, such as less than about 5 L/min. The present device 1 is especially suitable for measuring water at low rates. During a measurement process water may be restricted from flowing out of the receptacle 200, that is the output flow rate from the receptacle 200 may be less than the input flow rate, in some instances the output flow may be stopped, i.e., the output flow rate may be 0 L/min, during a measurement process. The output flow rate may be greater than 0 L/min during a measurement process. Water received in the receptacle 200 may be received at various flow rates depending on the device from which used water is received. The input and output flowrates may be controlled by valves provided upstream and downstream respectively to the receptacle 200.

The white light emitting element 100 may be provided at a first location 202 of the receptacle 200. The first location 202 may correspond to a substantially optically transparent portion of the at least one wall 201 of the receptacle 200.

The infra-red light emitting element 101 may be provided at a second location 203 of the receptacle 200. The second location 203 may correspond to a substantially optically transparent portion of the at least one wall 201 of the receptacle 200.

The light receiving element 102 may be provided at a third location 204 of the receptacle 200. The third location 204 may correspond to a substantially optically transparent portion of the at least one wall 201 of the receptacle 200.

Each of the white light emitting element 100, infra-red light emitting element 101 and light receiving element 102 may each be provided at respective locations 202, 203, 204 of the receptacle 200 corresponding to substantially optically transparent portions of the at least one wall 201. Optically transparent as used herein means that light in the visible and infra-red wavelength spectrums may pass through without a substantial reduction in intensity. Each of the first 202, second 203, and third 204 locations may be substantially provided as regular wall portions. If the receptacle 200 is tubular then the 202, second 203, and third 204 locations may be provided at radial points of the receptacle 200. That is, they may be radially opposed.

The receptacle 200, and in particular the at least one wall 201, may be manufactured from a polymer, such as a plastic, such as for example PET, PETG, PETE, PETT, and/or PMMA. The receptacle 200, may in some instances be manufactured from glass. The receptacle 200 may be moulded, such as blow moulded or injection moulded. The wall 201 thickness may be from about 1 mm to about 5 mm, a wall thickness of about 4 mm has been shown to display acceptable properties with respect to thermal isolation, wall 201 structure strength, and optical transparency. The entire device 1, including the receptacle 200 may be provided in a dark, environment. The device 1 may be encased or surrounded by an opaque housing to prevent ambient light entering the light receiving element 102.

The at least one wall 201 of the receptacle 200 has an internal surface 2010 and an external surface 2011. The internal surface 2010 is in contact with water received in the receptacle 200. The external surface 2011 is not in contact with water received in the receptacle 200. The internal surface 2010, and/or the external surface 2011 at each of the first location 202, second location 203, and third location 203 may be manufactured or processed such that it has a low surface roughness. The low surface roughness reduces scattering of emitted/received light. The low surface roughness at the internal surface 2010 is especially advantages in the present device 1 as it inhibits the formation and/or adhesion of a biofilm at the wall 201. As the device 1 is generally intended to measure the water quality of used water flowing at low flow rates, or zero flow rates, biofilm formation is a problem. Existing water quality measurement devices for measuring high flow-rate water in for example tubes or pipes are less susceptible to biofilm formation and often have relatively rougher surfaces. The low surface roughness may be achieved by polishing the first location 202, second location 203, or third location 204, after forming the receptacle 200. Ideally, the low surface roughness is achieved during forming, for example, moulding the receptacle 200 with specific low surface roughness regions and/or inserts to reduce surface roughness at the first, second and third locations 202, 203, 204.

In some instances, a spray element may be provided to the device 1 to spray at least one stream of water at the internal surface 2010 at the first location 202, the second location 203 and/or the third location 204. Ideally, and generally, the spray element sprays at least one stream of water at each of the first 202, second 203, and third surfaces 204. The stream of water cleans the internal surface 2010 of debris, biofilm, etc and improves the performance of the device 1. By providing the white light emitting element 100, the infra-red light emitting element 101, and the light receiving element 102 at substantially planar walls the cleaning is improved. Especially when compared to prior-art solutions with irregular protrusions designed to be installed into a water receptacle.

As described above, the receptacle 200 may be a tubular tank or tube, having a regular cylindrical wall 201. The tubular tank may have a diameter greater than the diameter of inlet tubing/piping etc, such that water velocity is reduced within the receptacle 200 compared to the inlet velocity. The tubular tank may have a diameter greater than the diameter of outlet tubing/piping, such that the flow velocity is reduced within the receptacle 200, compared to the flow velocity of water which has exited the receptacle. The regular cylindrical wall is also ideal for spray cleaning and does not have corners where fouling may build up.

An additional advantage of providing the white light emitting element 100, infra-red light emitting element 101, and the light receiving element 102 external to the receptacle 200 is that the flow of fluid within the receptacle 200 is not disturbed.

The white light emitting element 100, infra-red light emitting element 101, and/or the light receiving element 102 may be provided within a housing for securing the elements. Ideally, the white light emitting element 100, infra-red light emitting element 101, and the light receiving element 102 are provided as surface mounted elements on a single PCB. The PCB comprising a central aperture for receiving the receptacle 200. If the receptacle 200 is a tube, then the central aperture may be circular. With such an arrangement, the device 1 is installable to existing tubes having a sufficiently optically transparent wall 201. That is, the device 1 may be retrofitted to existing tubes. The elements 100, 102 are arranged to abut the receptacle 200, or ideally the sleeve 300 to reduce any potential light leakage.

The receptacle 200 itself may be receive the light emitting and receiving elements 100a, 100b, 101a, 101b, 102a, 102b. The receptacle 200 may therefore be custom manufactured for receiving the elements. The external surface 2011 of the wall 201 of the receptacle 200 may comprises a plurality of separate slots for receiving the white light emitting element 100, infra-red light emitting element 101, and/or the light receiving element 102. Each slot may be defined by a pair of opposing protruding ridges. Each of the slots may be located at the first 202, second 203, and third 204 locations respectively. The slots hold the white light emitting element 100, infra-red light emitting element 101, and light receiving element 102 in place at the wall 201 and ease installation and alignment.

A process for measuring the quality of water comprises: providing the device 1 as described herein; providing water to the receptacle 200; emitting white light from the white light emitting element 100 such that white light is received at the light receiving element 102, emitting infra-red light from the infra-red light emitting element 101 such that reflected or refracted infra-red light is received at the light receiving element 102; and, measuring the intensity of received white light and infra-red light at the light receiving element 102. After receiving the emitted infra-red and white light, the process may comprise comparing the measured received intensity of light at the light receiving element 102 to reference values corresponding to clean, non-processed, water.

The water quality measurement device 1 as described herein is capable of determining water colour and/or water turbidity. Ideally, the device 1 is capable of measuring both water colour and water turbidity. The water quality measurement device 1 is ideal for measuring the water quality of wash water from a dishwasher, washing machine, water recycling device, or other household or industrial device at which water is processed leading to changes to water quality. As the white light emitting element 100, infra-red light emitting element 101, and light receiving element 102 are arranged external the receptacle 200 the wash water does not contact the elements 100, 101, 102.

The combination of the white light emitting element 100 and the infra-red light emitting element 101 enables improved detection of water quality parameters of a fluid, and increases the potential to compensate for changes at the wall 201 of the receptacle 200. By separately increasing the intensity of light emitted by the white light emitting element 100 and/or the infra-red light emitting element 101 and measuring the corresponding change in received light at the light receiving element 102 the condition of the receptacle 200 may be investigated. For example, if increasing the intensity of light emitted from the white light emitting element 100 increases the intensity of light received at the light receiving element 102, this indicates that the portions 202, 204 of the wall 201 are substantially free from fouling. If, in contrast, increasing the intensity of light emitted from the infra-red light emitting element 101 does not result in an increase in intensity measured or detected at the light receiving element 102 then at least one of the portions 203, 204 may be fouled. The results of separate increases/decreases in intensity may be compared to determine where the fouling is present. The results of the comparison may be used for calibration such that an offset and/or calibration factor may be provided to the detected results for ultimately determining water quality.

The water quality measurement device 1 may comprise a temperature sensor for measuring the temperature of fluid. The temperature sensor may enable calibration of the measured optical data received at the light receiving element 102.

The light receiving element 102 may be configured to detect light for a duration of about 20 ms to about 100 ms. Ideally the light receiving element 102 is configured to detect light for about 50 ms. However, the exact duration of light detection, the integration time, is determined precisely via off-line calibration. A reduced duration of detection reduces the time required to measure a water quality parameter.

The device 1 may be calibrated off-line and/or on-line. Off-line calibration refers to a calibration process with a water having a known turbidity, as close to 0 NTU as possible. On-line calibration refers to a calibration process performed during every measurement process, where the water turbidity and/or colour is to be measured.

Off-line calibration comprises the steps: determining a suitable light receiving element 102a, 102b integration time, determining a suitable light emitting element 100a, 100b, 101a, 101b intensity, and determining baseline effective turbidity, and optionally baseline colour transmissivity.

The light receiving element 102a, 102b integration time is iteratively determined by first setting the intensity of a white light 100a, 100b, infra-red light 101a, 101b, emitting element to its maximum and the initial integration time to the minimum, i.e., shortest value. Then the measurements are performed with successively longer integration times. This process is repeated until the receiving element reading saturates, that is, reaches its maximum value. The process is performed separately for each white light emitting element 100a, 100b and infra-red light emitting element 101a, 101b, and light receiving element 102a, 102b. The integration time that results in saturation is the maximum light emitting element intensity which is then used for all subsequent measurements. This process is performed as the steps between integration times of the light receiving element are typically quite coarse, and ensures that the device has the ability to vary the brightness of the light emitting elements 100a, 100b, 101a, 101b giving a reading in the entirety of the light receiving element's 102a, 102b linear range.

The intensity of light emitted from the white light emitting element 100, and the infra-red light emitting element 101 is set to be about 75% or lower than the maximum intensity. Such an intensity provides a suitable dynamic range and avoid saturating the light receiving element 102. By calibrating and adapting the integration time of the light receiving element 102 and the intensity of the white light and infra-red light emitting elements 101 the device is robust against variations in light emitting element 100, 101 intensity and light receiving element 102 sensitivity, device variations, component wear and fouling of the receptacle 200 wall 201.

The baseline effective turbidity is determined by performing a measurement with clear, non-processed water i.e., water having a known turbidity ideally as close to 0 NTU as possible. The white light emitting element 100 emits light which is received at the light receiving element 102. Separately, that is not simultaneously, the infra-red light emitting element 101 emits light which is received at the light receiving element 102. The measurement may be performed with the at least two white light emitting elements and at least two infra-red light emitting elements, providing the terms denoted β_{B0} and β′_{A0} from the above equations. The detected values provide a net diffractive reading being

$${β}_{B , net}^{′} = {β}_{B} - {β}_{B 0}$$

$$and$$

$${β}_{A , net}^{′} = {β}_{A}^{′} - {β}_{A 0} .$$

A similar process is performed to determine the baseline colour, RGB, transmissivity.

The detected values are converted to industry standard NTU units to provide a measurement unit which is comparable to other devices used within the field of water turbidity measurement.

The on-line calibration process comprises emitting white light from the white light emitting element 100, emitting infra-red light from the infra-red light emitting element 101, and detecting and measuring the light received at the light receiving element 102. The on-line calibration process also comprises measuring the light received at the light receiving element 102 with the white light emitting element 100, and the infra-red light emitting element 101 not emitting light, i.e., a dark-level reading. The dark-level reading is subtracted from the measured value when the white 100 and infra-red 101 were emitting light. This provides a net value removing any constant background illumination.

A water recycling system may comprise the water quality measurement device 1. The water recycling system has improved water recycling properties due to the improved measurement performance provided by the device 1. The water recycling device may be configured to recycle or discard water based on the measured water quality.

A water consumption device such as a washing machine, or dishwasher may comprise the water quality measurement device 1. The receptacle 200 of the device 1 receives wash water for measurement. The water consumption device may be configured to reuse or discard processed water based on the measured water quality. The water quality measurement device 1 is especially suitable for use with processed washing machine water as such water may be coloured due to dyes/pigments from the washed fabric soiling the processed wash water.

### EXPERIMENTAL SECTION

### Experiment 1 Measurement of Water Turbidity at Different Turbidity Levels with Water Quality Measurement Device

The water quality measurement device 1 comprising a receptacle 200 was prepared as described above. Samples of water turbidity at 13 different levels were prepared. The 13 turbidity levels ranged from 0 to 600 Nephelometric Turbidity Units (NTU). The NTU values for each of the samples were determined for each of the reference samples with a calibration unit, Apera TN400 Tubidity Meter (2021). The calibration unit was itself calibrated with ISO 7027 Clear polymer standard solutions at 0.00, 20.0, 100, 400, 800 NTU respectively (Apera).

Infra-red light was emitted from the infra-red light emitting element 101 at a specific intensity, and over a specific duration. Infra-red light was detected at the light receiving element 102 for a specific measurement duration.

99 473 measurements were performed of the 13 test samples. The device 1 displayed excellent spot on repeatability for all samples. The device 1 displayed a Level of Detection (LOD) of 0.04 NTU, and a level of quantification (LOQ) of 0.13 NTU.

### Experiment 2 Measurement of Water Colour at Different Dye Levels with Water Quality Measurement Device

The water quality measurement device 1 comprising a receptacle 200 was prepared as described herein. 10 sample solutions were prepared with a concentration of black clothing dye (Dylon Renovator Black). Concentrations were 0, 0.1, 0.4, 0.7, 1.3, 1.9, 3.3, 16.3, 29.0, 42.9 mg/L respectively.

White light was emitted from the white light emitting element 100 and received at the light receiving element 102 for specific emission and measurement durations.

26100 measurements were performed with excellent repeatability. The mean performance of the three Red, Green and Blue (RGB) channels was determined. The device 1 displayed a mean LOD of 33 μg/L, and a LOQ of 110 μg/L.

Although, the present invention has been described above with reference to specific embodiments, it is not intended to be limited to the specific form set forth herein. Rather, the invention is limited only by the accompanying claims.

In the claims, the term “comprises/comprising” does not exclude the presence of other elements or steps. Furthermore, although individually listed, a plurality of means, elements or method steps may be implemented by e.g., a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality. Reference signs in the claims are provided merely as a clarifying example and shall not be construed as limiting the scope of the claims in any way.

1-19. (canceled)

20. A water quality measurement device for measuring the water quality of processed wash water, the water quality measuring device comprising:
a white light emitting element configured to emit substantially white light,
an infra-red light emitting element configured to emit substantially infra-red light, and
a light receiving element configured to receive and detect light from the white light emitting element and the infra-red light emitting element;
wherein the white light emitting element is arranged co-axial and opposite the light receiving element, and wherein the emission axis of the infra-red emitting element is arranged substantially orthogonal the incident axis of the light receiving element.

21. The water quality measurement device according to claim 20, wherein the device comprises a receptacle for receiving a volume of water, and wherein each of the white light emitting element, the infra-red light emitting element, and the light receiving element are external to and directed into the receptacle.

22. The water quality measurement device according to claim 21, wherein the receptacle is defined by at least one wall, and wherein the white light emitting element, the infra-red emitting element, and the light receiving element are separated from the volume of water during measurement by the at least one wall of the receptacle.

23. The water quality measurement device according to claim 20, wherein the device comprises at least two white light emitting elements, at least two infra-red light emitting elements and at least two light receiving elements, and wherein each of the white light emitting elements are substantially co-axial and opposite a respective light receiving element, and wherein each of the infra-red light emitting elements are arranged substantially orthogonal the incident axis of a respective light receiving element.

24. The water quality measurement device according to claim 23, wherein a first white light emitting element is co-located with a second infra-red light emitting element, and wherein a second white light emitting element is co-located with a first infra-red light emitting element.

25. The water quality measurement device according to claim 23, wherein the at least two light receiving elements are arranged such that their respective light incidence axes are substantially orthogonal.

26. The water quality measurement device according to claim 21, wherein the at least one wall of the receptacle is a tubular wall, and wherein the at least one white light emitting element, the at least one infra-red light emitting element, and the at least one light receiving element are provided around the circumference of the tubular wall.

27. The water quality measurement device according to claim 21, wherein the device comprises an optically opaque sleeve surrounding the wall, the sleeve having a plurality of slots corresponding to emission and incidence axes for the at least one white light emitting element, the at least one infra-red light emitting element, and the light receiving element.

28. The water quality measurement device according to claim 27, wherein the sleeve abuts the at least one wall of the receptacle.

29. The water quality measurement device according to claim 27, wherein the sleeve comprises at least four slots, wherein each slot is aligned and opposite a corresponding slot.

30. The water quality measurement device according to claim 20, wherein the water quality measurement device is configured to detect the turbidity and/or colour of the water.

31. The water quality measurement device according to claim 20, wherein the light receiving element is a light sensor configured to separately detect light in wavelengths corresponding to red, green, blue, and white light.

32. The water quality measurement device according to claim 20, wherein the water is processed wash water from a washing machine, dishwasher and/or water recycling device.

33. The water quality measurement device according to claim 20, wherein water is received into the receptacle via an inlet and exits the receptacle via a separate outlet.

34. A water recycling device comprising the water quality measurement device according to claim 20, wherein the water recycling device receives processed wash water from a washing machine and/or dishwasher, and determines whether water should be recycled or discarded based on the measured water quality of the processed wash water.

35. A process for measuring the quality of water, comprising:
providing a device for measuring water quality according to claim 21
providing water to the receptacle,
emitting white light from at least one white light emitting element such that white light is received at the at least one light receiving element,
emitting infra-red light from the at least one infra-red light emitting element such that reflected or refracted infra-red light is received at the at least one light receiving element
measuring the intensity of received white light and infra-red light at the at least one light receiving element.

36. The process for measuring water quality according to claim 35, comprising comparing the measured received intensity of light at the at least one light receiving element to reference values corresponding to clean, non-processed, water.

37. The process for measuring water quality according to claim 35, wherein the process comprises:
providing said device comprising at least two white light emitting elements, at least two infra-red light emitting elements and at least two light receiving elements, and wherein each of the white light emitting elements are substantially co-axial and opposite a respective light receiving element, and wherein each of the infra-red light emitting elements are arranged substantially orthogonal the incident axis of a respective light receiving element,
comparing the light from the at least two white light emitting elements received at each light receiving element respectively, and
comparing the light from the at least two infra-red light emitting elements received at each light receiving element respectively.

38. The process for measuring water quality according to claim 36, wherein the reference values corresponding to clean, non-processed water are generated by a calibration process, the calibration process comprising:
providing non-processed water, the water having a low turbidity, to the measurement device,
determining a suitable light receiving element integration time by setting the intensity of the white light emitting element and the infra-red light emitting element to a maximum intensity,
initially setting the integration time of the light receiving element to a minimum value,
measuring the intensity of light received at the light receiving element from the white light emitting element,
measuring the intensity of the light received at the light receiving element from the infra-red light emitting element,
repeating the measurements with a successively increasing integration time until the light receiving element reaches saturation point, thereby determining the saturation point, and
setting the integration time of the light receiving element to the integration time corresponding to the saturation point,
measuring the baseline turbidity of the water by emitting white light from the white light emitting element and infra-red light emitting element and determining the intensity of received light at the light receiving element, thereby generating the reference values.
